(19) United States
(12) Patent Application Publication
(10) Pub. No.: US 2018/0177748 A1
(43) Pub. Date: Jun. 28, 2018

(54) COMBINATION MEDICATION FOR NEURO-DEGENERATIVE DISEASES

(71) Applicants: Cary Erwin FECHTER, North Charleston, SC (US); Nancy Sharon KEDEM, Melville, NY (US); Douglas F. CRANE, Charleston, SC (US); Thomas James THOMAS, Summerville, SC (US)

(72) Inventors: Cary Erwin FECHTER, North Charleston, SC (US); Nancy Sharon KEDEM, Melville, NY (US); Douglas F. CRANE, Charleston, SC (US); Thomas James THOMAS, Summerville, SC (US)

(21) Appl. No.: 15/738,357

(22) PCT Filed: Jun. 20, 2016
(86) PCT No.: PCT/US2016/038351

§ 371 Date: Dec. 20, 2017

Related U.S. Application Data
(63) Continuation of application No. 14/745,400, filed on Jun. 20, 2015, now abandoned.

(51) Int. Cl. A61K 31/145; A61K 31/343; A61K 45/06; A61K 31/135; A61K 31/137; A61K 31/445; A61K 31/165

(52) U.S. Cl. CPC A61K 31/145; A61K 31/343; A61K 45/06; A61K 31/135; A61K 2300/00; A61K 31/445; A61K 31/165; A61K 31/137

(57) ABSTRACT

A pharmaceutical composition containing a benzhydrylsulfinylacetamide antidepressant, and either or both of an SSRI uptake inhibitor and/or a stimulating antidepressant has been shown to be effective in ameliorating symptoms of dementia, including those forms of dementia diagnosed as Alzheimer's disease.

### BACKGROUND OF THE INVENTION

### 1. Field of the Invention

The invention pertains to pharmaceutical compositions useful for treatment of multiple symptoms of Alzheimer's disease and dementia, and to the use thereof for these purposes.

### 2. Description of the Related Art

Dementia is a common characteristic associated especially with increasing age, and is sometimes referred to as “senility.” It is characterized by long term and generally gradual impairment of the ability to think and remember that is great enough to affect a persons daily functioning. Other common symptoms include emotional problems, decrease in language skills, paranoia, and decreased motivation.

In 1906 Dr. Alois Alzheimer first recognized the disease now bearing his name. He described the disease as a dementia characterized by severe memory loss and confusion with pathological changes to neurons. Frequency of diagnosis increased with the implementation of a cognitive measurement scale in 1968 and currently Alzheimer's disease is the most widely diagnosed form of dementia. Understanding of the molecular basis of the disease improved with the discovery of the beta amyloid and tau proteins that form the toxic deposits causing neuron death. In spite of this, no curative agents have been developed although several acetylcholinesterase antagonists have been approved for treating memory deterioration. These drugs are currently being used with limited results.

Common treatments for dementia include the use of antidepressants and anti-anxiety medications. However, these have proven to be only of marginal effectiveness as compared with placebos. Treatment for Alzheimer's disease is much less developed, as only somewhat recently were animal models proposed to test drug efficacy. A great deal of developmental work has centered on slowing the course of the disease. Little progress has been made in alleviating symptoms such as reduced cognitive behavior.

It would be desirable to provide a pharmaceutical composition which is effective in improving the daily functioning of patients suffering from dementia in general, and also in patients diagnosed with Alzheimer's disease.

### SUMMARY OF THE INVENTION

It has now been surprisingly and unexpectedly discovered that a therapy employing a combination of a benzhydrylsulfinylacetamide wakefulness promoting agent and either or both of a selective serotonin reuptake inhibitor (SSRI) and a stimulating antidepressant, and optionally a cholinesterase inhibitor, is effective in reducing symptoms associated with dementia.

### DETAILED DESCRIPTION OF THE PREFERRED EMBODIMENTS

Our research has revealed that two symptoms associated with Alzheimer's disease as well as other neurodegenerative diseases but not addressed by current therapeutic regimens, specifically depression and overall abnormal sleep patterns, can have a significant negative effect on the quality of life of patients. The abnormal sleep patterns in particular, by disrupting normal circadian rhythm, exacerbate the confusion, loss of mental focus, and feelings of disorientation characteristic of the disease. Furthermore, this abnormal circadian pattern of daytime sleepiness combined with restlessness and inability to sleep properly at night (sometimes referred to as “sundowning”) greatly complicates the efforts of caregivers.

Including treatment for these two symptoms in the overall treatment protocol can improve memory and patient quality of life as much as the drugs currently approved for memory deterioration.

Since traditional sleep regulating drugs such as tricyclics and benzodiazepines can increase the lethargy, confusion, and forgetfulness, already present in Alzheimer's patients, a different therapeutic approach is needed. Specifically, drugs of the benzhydrylsulfonylamide type can reduce excessive daytime sleepiness and promote normal circadian rhythm while avoiding the side effects of traditional sleep regulating drugs.

Combining this therapy with administration of either or both of a Selective Serotonin Reuptake Inhibitor (SSRI) antidepressant and a stimulating antidepressant such as bupropion reduces the severity of associated clinical depression symptoms. SSRI's also increase alpha secretase activity, which has been shown to aid in clearing amyloid beta, one of the peptides associated with formation of damaging plaques in the brain.

The overall results of this combination approach in conjunction with traditional acetylcholinesterase antagonist therapy are improvement in patient cognition including responsiveness and attitude toward their environment.

Additionally, combining all of the active pharmaceutical agents in a single formulation ensures that the timing of administration of the drugs is optimal. Since Alzheimer's caregivers typically have multiple duties to their patients, the single dosage format simplifies caregiver routine and improve compliance with the treatment regimen.

Benzhydrylsulfinylacetamides are known, as described in Lafon EP 0097071, and correspond to the general formula

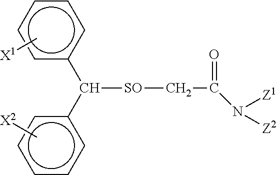

wherein
X¹ and X² may be the same or different, and are H, Cl, or F;
Z¹ and Z² may be the same or different and are H, —CH₃, —CH(CH₃)₂, or —C(CH₃)₃, with the proviso that at least one of Z¹ and Z² is H.

Preferably, both Z¹ and Z² are H, and also preferably one of the following holds: X¹ and X² are both H, one of X¹ and X² is H and the other of X¹ and X² is 4-Cl or 4-F, or both X¹ and X² are 4-F. Most preferably, both Z¹ and Z² are H and both X¹ and X² are H, in other words, benzhydrylsulfinylacetamide itself (CAS 68693-11-8). The benzhydrylsulfinylacetamides may be used in racemic form, or in the form of their fully or partially resolved optical isomers wherein one isomer, preferably the levorotatory form, is present in enantiomeric excess. Racemic benzhydrylsulfinylacetamide is available commercially as PROVIGIL® from Cephalon, Inc., and is known pharmaceutically as modafinil.

Selective serotonin reuptake inhibitors (SSRIs), also known as “serotonin-specific reuptake inhibitors” increase the extracellular concentration of the neurotransmitter serotonin. The action of SSRIs may be contrasted with serotonin reuptake inhibitors (SRIs), as the latter also greatly affect reuptake of dopamine and norepinephrine. SSRIs primarily affect serotonin reuptake, with lesser effect on the two other neurotransmitters, e.g. are “selective” or “specific” relative to reuptake of serotonin. By preventing serotonin reuptake, believed to be caused by minimizing reabsorbtion of serotonin into presynaptic cells, extracellular serotonin levels are increased.

Those skilled in the pharmaceutical arts are aware of numerous SSRIs which can be used. Unlike the benzhydrylsulfinylacetamides, the SSRIs span a wide range of chemical structures which share little in common except their serotonin selectivity. Useful SSRIs and/or their salts include citalopram (CAS 59729-33-8), escitalopram (CAS 128196-01-1), fluoxetine (CAS 54910-89-3), fluvoxamine (CAS 56296-78-7), paroxetine (CAS 78246-49-8), and sertraline (CAS 79617-96-2). Preferred are citalopram, escitalopram, fluoxetine, paroxetine, sertraline. More preferred are citalopram and sertraline. Sertraline is most preferred.

Stimulating antidepressants as defined therein are dopamine and norepinephrine reuptake inhibitors, and are associated with a decrease in depression symptoms along with an increased energy level. Bupropion is a stimulating antidepressant, and is preferred. Stimulating depressants as used herein do not include SSRIs, which have little effect on dopamine or norepinephrine reuptake.

A cholinesterase inhibitor is an optional ingredient. Cholinesterase inhibitors are known, examples of which include donepezil (CAS 120014-06-4), galantamine, and rivastigmine. Donepezil is preferred. The cholinesterase inhibitor may include more than one such inhibitor. Examples of combinations include donepezil and galantamine, donepezil and rivastigmine, donepezil, galantamine, and rivastigmine, and galantamine and rivastigmine.

The CAS numbers previously given may be those of the active ingredient base compound itself, or a pharmaceutically acceptable salt thereof. Those skilled in the art are cognizant of numerous counterions which can be used in such pharmaceutically acceptable salts. These salts are used in particular to alter the solubility or solubility profile of the particular compound. Suitable salts, e.g., are the hydrohalide salts such as hydrochlorides and hydrobromides, acetates, propionates, maleates, oxalates, bezylates, nitrates, sulfates, phosphates, and tartrates. This list is exemplary and not limiting. Further examples of pharmaceutically acceptable salts may be found in P. H. Stahl and C. G. Wermuth, Handbook of Pharmaceutical Salts: Properties, Wiley-VCH, Weinheim, 2002.

The amounts of each individual ingredient are therapeutically effective amounts, which can be varied depending on the individual patient's body chemistry. The ingredients are preferably contained in a single dosage, for example a pill, capsule, lozenge, gel cap, etc. The therapeutically effective amounts can be determined in a conventional manner, for example using double blind testing. In no case should any ingredient be present in an amount generally considered toxic.

As guidance to one skilled in the arts of pharmaceutical compounding and clinical trials, the benzhydrylsulfinylacetamide dosage is preferably in the range of 50-500 mg, more preferably 100-300 mg. A dosage of 200 mg of modafinil has been found to be particularly effective. Other benzhydrylsulfinylacetamides can be used in similarly effective amounts, meaning that compounds from this class which are more active may be used in correspondingly lesser amounts, and vice versa.

The SSRI antidepressant is preferably used in amounts of from 5 to 250 mg, more preferably 50-200 mg, and most preferably, 75-150 mg. A dosage of 100 mg of sertraline has been found to be particularly effective. SSRIs which are more active than sertraline can be used in lesser quantities, and vice versa. Citalopram is preferably used in amounts of from 5-50 mg.

The cholinesterase inhibitor, when included, is preferably used in amounts of from 1.5 to 20 mg, more preferably 3-15 mg, yet more preferably 3-10 mg, and most preferably from 3-8 mg. A dosage of 5 mg of donepezil has been shown to be particularly effective. For cholinesterase inhibitors with greater activity, correspondingly lesser dosages can be used, and vice versa.

The stimulating antidepressant is preferably bupropion, and when included, is preferably used in amounts of 20 mg to 400 mg, more preferably 50-250 mg, and most preferably 75-200 mg. A dosage of 150 mg has been proven to be particularly effective. For stimulating antidepressants of higher activity, correspondingly lesser amounts may be used, and vice versa.

Each of the ranges given for each of the flour classes of ingredients, benzhydrylsulfinylacetamides, SSRI inhibitors, cholinesterase inhibitors, and stimulating depressants, may be used in conjunction with any of the ranges of the other classes of ingredients. This allows the formulation to be altered for specific patients or for specific classes of patients with similar body chemistry. Moreover, each start point and end point of each range for any one class of ingredients may be combined with an end point or start point of another disclosed range for the same ingredient or ingredient class to define another range.

The quantities given above are intended for simultaneous administration once per day. If administration is to be performed more than once per day, it is preferred that the dosages be lowered. It is preferable that administration be once per day in the morning. A second dose at midday or in the early late afternoon may also be used. It is not preferable that administration take place in late afternoon or evening, as the ability to achieve a restful night's sleep may be impaired.

The compositions of the subject invention may be formulated in liquid, gel, or solid form, using conventional pharmaceutical excipients. Solid dosage forms are preferred. Non-limiting examples of suitable excipients include fillers, examples of which are dextrose, sucrose, starches, and calcium carbonate; tableting aids such as metal stearates; coatings such as polyvinylpyrrolidone, polyvinylalcohol, and crosslinked and uncrosslinked gelatin; thickeners such as modified celluloses, for example carboxymethyl cellulose; natural oils and modified natural oils such as fatty acid esters, olive oil, fish oil, liquid and solid triglycerides, and the like. Suitable pharmaceutical excipients may be found in numerous treatises, for example, A H. Kibbe, Handbook of Pharmaceutical Excipients, APhA publications, © 2000.

The symptom relieving effects of the claimed compositions was evaluated by clinical observation of patients exhibiting symptoms of dementia, including patients diagnosed with Alzheimer's disease.

### EXAMPLES

### Example 1

A 50 year old female with forgetfulness, previously diagnosed with mild Alzheimer's disease by a neurologist was evaluated by questionnaire using the Epworth Sleepiness Scale (ESS) and found to be exhibiting excessive daytime sleepiness. Patient responses were consistent with clinical depression using the Beck Depression Scale (BDS) as characterized in the Diagnostic and Statistical Manual, fifth edition (DSM5).

A treatment protocol consisting of simultaneously administering 200 mg. Modafinil, 100 mg. Sertraline, and 5 mg. Donepezil once per day in the morning was initiated.

After six weeks the patient was re-evaluated by questionnaire using the ESS and found to be exhibiting a more normal sleep pattern. Patient responses demonstrated lessened depression criteria using BDS/DSM5.

Patient described overall attitude as more positive.

### Example 2

A 65 year old female with forgetfulness, previously diagnosed with moderate-severe Alzheimer's disease by a neurologist was evaluated through caregiver responses using the ESS and found to be exhibiting excessive daytime sleepiness.

Caregiver responses to standard questionnaire provided positive indication of clinical depression using BDS/ DSM5.

A treatment protocol consisting of simultaneously administering 200 mg. Modafinil, 100 mg. Sertraline, and 5 mg. Donepezil once per day in the morning was initiated.

After six weeks the patient was re-evaluated by questionnaire through caregiver's responses using the ESS and found to be exhibiting a more normal sleep pattern. Caregiver responses demonstrated lessened patient depression criteria using BDS/DSM5.

Caregiver described patient's overall attitude as more positive.

### Example 3

A 55 year old male with forgetfulness, previously diagnosed with mild Alzheimer's disease by a neurologist was evaluated by questionnaire using the Epworth Sleepiness Scale (ESS) and found to be exhibiting excessive daytime sleepiness. Patient responses were consistent with clinical depression using the Beck Depression Scale (BDS) as characterized in the Diagnostic and Statistical Manual, fifth edition (DSM5).

A treatment protocol consisting of simultaneously administering 200 mg. Modafinil, 100 mg. Sertraline, and 5 mg. Donepezil once per day in the morning was initiated.

After six weeks the patient was re-evaluated by questionnaire using the ESS and found to be exhibiting a more normal sleep pattern. Patient responses demonstrated lessened depression criteria using BDS/DSM5

Patient described overall attitude as more positive.

### Example 4

A 71 year old male with forgetfulness, previously diagnosed with moderate-severe Alzheimer's disease by a neurologist was evaluated through caregiver responses using the ESS and found to be exhibiting excessive daytime sleepiness.

Caregiver responses to standard questionnaire provided positive indication of clinical depression using BDS/ DSM5.

A treatment protocol consisting of simultaneously administering 200 mg. Modafinil, 100 mg. Sertraline, and 5 mg. Donepezil once per day in the morning was initiated.

After six weeks the patient was re-evaluated by questionnaire through caregiver's responses using the ESS and found to be exhibiting a more normal sleep pattern. Caregiver responses demonstrated lessened patient depression criteria using BDS/DSM5.

Caregiver described patient's overall attitude as more positive.

### Example 5

A 53 year old female with forgetfulness, previously diagnosed with mild Alzheimer's disease by a neurologist was evaluated by questionnaire using the Epworth Sleepiness Scale (ESS) and found to be exhibiting excessive daytime sleepiness. Patient responses were consistent with clinical depression using the Beck Depression Scale (BDS) as characterized in the Diagnostic and Statistical Manual, fifth edition (DSMS).

A treatment protocol consisting of simultaneously administering 200 mg. Modafinil, 100 mg. Sertraline, 5 mg. Donepezil, and 150 mg. Bupropion once per day in the morning was initiated.

After six weeks the patient was re-evaluated by questionnaire using the ESS and found to be exhibiting a more normal sleep pattern. Patient responses demonstrated lessened depression criteria using BD S/DSM5.

Patient described overall attitude as more positive.

### Example 6

A 65 year old female with forgetfulness, previously diagnosed with moderate-severe Alzheimer's disease by a neurologist was evaluated through caregiver responses using the ESS and found to be exhibiting excessive daytime sleepiness.

Caregiver responses to standard questionnaire provided positive indication of clinical depression using BDS/ DSMS.

A treatment protocol consisting of simultaneously administering 200 mg. Modafinil, 100 mg. Sertraline, 5 mg. Donepezil, and 150 mg. Bupropion once per day in the morning was initiated.

After six weeks the patient was re-evaluated by questionnaire through caregiver's responses using the ESS and found to be exhibiting a more normal sleep pattern. Caregiver responses demonstrated lessened patient depression criteria using BDS/DSMS.

Caregiver described patient's overall attitude as more positive.

Example 7: A 50 year old male with forgetfulness, previously diagnosed with mild Alzheimer's disease by a neurologist was evaluated by questionnaire using the Epworth Sleepiness Scale (ESS) and found to be exhibiting excessive daytime sleepiness. Patient responses were consistent with clinical depression using the Beck Depression Scale (BDS) as characterized in the Diagnostic and Statistical Manual, fifth edition (DSM5).

A treatment protocol consisting of simultaneously administering 200 mg. Modafinil, 100 mg. Sertraline, 5 mg. Donepezil, and 150 mg. Bupropion once per day in the morning was initiated.

After six weeks the patient was re-evaluated by questionnaire using the ESS and found to be exhibiting a more normal sleep pattern. Patient responses demonstrated lessened depression criteria using BDS/DSM5.

Patient described overall attitude as more positive.

### Example 8

A 72 year old male with forgetfulness, previously diagnosed with moderate-severe Alzheimer's disease by a neurologist was evaluated through caregiver responses using the ESS and found to be exhibiting excessive daytime sleepiness.

Caregiver responses to standard questionnaire provided positive indication of clinical depression using BDS/ DSM5.

A treatment protocol consisting of simultaneously administering 200 mg. Modafinil, 100 mg. Sertraline, 5 mg. Donepezil, and 150 mg. Bupropion once per day in the morning was initiated.

After six weeks the patient was re-evaluated by questionnaire through caregiver's responses using the ESS and found to be exhibiting a more normal sleep pattern. Caregiver responses demonstrated lessened patient depression criteria using BDS/DSM5.

Caregiver described patient's overall attitude as more positive.

As required, detailed embodiments of the present invention are disclosed herein; however, it is to be understood that the disclosed embodiments are merely exemplary of the invention that may be embodied in various and alternative forms. The figures are not necessarily to scale; some features may be exaggerated or minimized to show details of particular components. Therefore, specific structural and functional details disclosed herein are not to be interpreted as limiting, but merely as a representative basis for teaching one skilled in the art to variously employ the present invention.

While exemplary embodiments are described above, it is not intended that these embodiments describe all possible forms of the invention. Rather, the words used in the specification are words of description rather than limitation, and it is understood that various changes may be made without departing from the spirit and scope of the invention. Additionally, the features of various implementing embodiments may be combined to form further embodiments of the invention.

1. A pharmaceutical composition, comprising:
a) a benzhydrylsulfinylacetamide wakefulness promoting agent; and
b) a cholinesterase inhibitor.

2. The pharmaceutical composition of claim 1 further comprising a selective serotonin reuptake inhibitor.

3. The pharmaceutical composition of claim 1 further comprising a stimulating antidepressant.

4. The pharmaceutical composition of claim 1, wherein the benzhydrylsulfinylacetamide wakefulness promoting agent has the formula 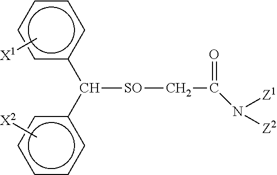
wherein
X¹ and X² may be the same or different, and are H, Cl, or F;
Z′ and Z² are the same or different and are H, —CH₃, —CH(CH₃)₂, or —C(CH₃)₃, with the proviso that at least one Z¹ and Z² is H, or a pharmaceutically acceptable salt thereof.

5. The pharmaceutical composition of claim 1, wherein the benzhydrylsulfinylacetamide wakefulness promoting agent is benzhydrylsulfinylacetamide, or a pharmaceutically acceptable salt thereof.

6. The pharmaceutical composition of claim 2, wherein the selective serotonin reuptake inhibitor is at least one of citalopram, escitalopram, fluoxetine, fluvoxamine, paroxetine, sertraline, and a pharmaceutically acceptable salt thereof.

7. The pharmaceutical composition of claim 1 further comprising a selective serotonin reuptake inhibitor and a stimulating antidepressant.

8. The pharmaceutical composition of claim 1, further comprising a pharmaceutically acceptable excipient.

9. The pharmaceutical composition of claim 1, wherein the cholinesterase inhibitor is at least one of donepezil, galantamine, rivastigmine, and a pharmaceutically acceptable salt thereof.

10. The pharmaceutical composition of claim 1 consisting essentially of the benzhydrylsulfinylacetamide wakefulness promoting agent, the cholinesterase inhibitor, and at least one of a selective serotonin reuptake inhibitor and a stimulating antidepressant.

11. The pharmaceutical composition of claim 3, wherein the stimulating antidepressant is a dopamine and norepinephrine reuptake inhibitor.

12. The pharmaceutical composition of claim 3, wherein the stimulating antidepressant is bupropion, or a pharmaceutically acceptable salt thereof.

13. The pharmaceutical composition of claim 2, wherein the benzhydrylsulfinylacetamide wakefulness promoting agent is present in an amount of 50-500 mg, and the selective serotonin reuptake inhibitor is sertraline present in an amount of 15-250 mg.

14. The pharmaceutical composition of claim 2, wherein the selective serotonin reuptake inhibitor is citalopram present in an amount of 5-50 mg.

15. The pharmaceutical composition of claim 3, wherein the stimulating antidepressant is present in an amount of 50-400 mg.

16. A method for alleviating symptoms associated with dementia, comprising administering a pharmaceutical composition comprising a benzhydrylsulfinylacetamide wakefulness promoting agent to a patient having dementia.

17. The method of claim 16, wherein the dementia is diagnosed as Alzheimer's disease.

18. A method for alleviating symptoms associated with abnormal circadian patterns, comprising administering a pharmaceutical composition comprising a benzhydrylsulfinylacetamide wakefulness promoting agent to a patient having abnormal circadian patterns.

19. The method of claim 18, wherein the patient exhibits at least one of at least one of daytime sleepiness, restlessness, and sundowning.

20. The method of claim 18, wherein the patient exhibits daytime sleepiness, restlessness, and sundowning.
